(19) United States
(12) Patent Application Publication
(10) Pub. No.: US 2003/0194711 A1
(43) Pub. Date: Oct. 16, 2003

(54) System and method for analyzing gene expression data

(76) Inventors: Matthew Zapala, La Jolla, CA (US); David Lockhart, Del Mar, CA (US); Carrolee Barlow, Del Mar, CA (US); Jennifer A. Greenhall, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/121,508

(22) Filed: Apr. 10, 2002

(51) Int. Cl. C12Q 1/68; G06F 19/00; G01N 33/48; G01N 33/50

(52) U.S. Cl. 435/6; 702/20

(57) ABSTRACT

A system and methods for identifying sequence diversity in a gene or expressed sequence is disclosed wherein hybridization differences arising from polymorphic bases in analogous expressed sequences are identified between two or more nucleotide populations. By scaling the hybridization data to account for differences in abundance and observed intensity, sequence diversity can be identified in a highly specific and sensitive manner. Data confidence levels are also accounted for to increase the accuracy of the sequence diversity determination. The invention can be applied to both newly collected gene expression data and archived data to generate valuable insight into polymorphic behavior within complex nucleotide populations.

### BACKGROUND OF THE INVENTION

1. Field of the Invention

The invention relates to the field of array-based gene expression analysis and more particularly to a system and method for determining sequence differences using oligonucleotide array gene expression data.

2. Description of the Related Art

Considerable time and energy has been expended to map whole genomes using high-throughput sequencing technologies. As a result, a complete genetic map now exists, or will exist in the near future, for a number of organisms including humans. Furthermore, gene expression analysis techniques have progressed to the point where it is now possible to simultaneously assess the relative abundance and changes in expression for many thousands of genes. As our understanding of complex biological processes increases through global assessment of genetic information, it has become apparent that numerous polymorphic variations, or areas of sequence diversity, exist within many genomes.

Available research data has shown that polymorphic variations may play a role in a number of disease states and their occurrence is more widespread than previously thought. Presently, the ability to efficiently determine where polymorphisms may be found in a genome is limited by the lack of suitable high-throughput methods for sequence diversity analysis. Conventional methods for identifying polymorphic variations are not designed to assess sequence diversity on a global scale and as a result the assessment of large amounts of genetic information related to polymorphic behavior can be impractical and time consuming.

Recently, array-based expression analysis platforms have become the preferred means for detecting and analyzing complex message populations, such as those found in the cells of biological organisms. Arrays present an attractive method for assessing gene expression on a global scale; however, like other expression analysis techniques, arrays have not been adapted for use in simultaneous gene expression analysis and polymorphism or sequence diversity analysis. Because of the widespread acceptance of array-based technologies as a standard for assessing gene expression, it would be desirable to incorporate within the analytical framework of these technologies the ability to simultaneously identify regions of sequence diversity.

One class of arrays commonly used in differential expression studies include microarrays or oligonucleotide arrays. These arrays utilize a large number of probes that are synthesized directly on a substrate and are used to interrogate complex RNA or message populations based on the principle of complementary hybridization. Typically, these microarrays provide sets of 16 to 20 oligonucleotide probe pairs of relatively small length (20mers-25mers) that span a selected region of a gene or nucleotide sequence of interest. The probe pairs used in the oligonucleotide array may also include perfect match and mismatch probes that are designed to hybridize to the same RNA or message strand. The perfect match probe contains a known sequence that is fully complementary to the message of interest while the mismatch probe is similar to the perfect match probe with respect to its sequence except that it contains at least one mismatch nucleotide which differs from the perfect match probe. During expression analysis, the hybridization efficiency of messages from a sample nucleotide population are assessed with respect to the perfect match and mismatch probes in order to validate and quantitate the levels of expression for many messages simultaneously.

Conventional expression analysis methodologies, such as those employing oligonucleotide arrays used in conjunction with templates derived from messenger RNA, have not been adapted for use in simultaneous polymorphic analysis. These methodologies fail to recognize that gene expression information from more than one sample population may be used to identify sequence variations between specific sequences within each population.

Although the expression arrays, including oligonucleotide arrays, were not originally designed to detect sequence differences, it would be desirable to combine the ability to identify changes in gene expression with the ability to detect and determine polymorphisms in a complex sample population using the same platform for analysis. A consolidated array analysis procedure as described above would provide a valuable complement to other methods, such as direct candidate sequencing and quantitative trait locus (QTL) analysis, for the identification of genes responsible for important phenotypes.

### SUMMARY OF THE INVENTION

In one embodiment the invention comprises a system for determining genetic differences between a first sample population and a second sample population. The components of the system include: a first data acquisition module, a second data acquisition module, a data comparison module, and a data analysis module. The first data acquisition module is configured to read first expression data from a first expression array contacted by the first sample population and a second data acquisition module configured to read second expression data from a second expression array contacted by the second sample population, wherein the first expression array and the second expression array comprise a plurality of oligonucleotide fragments of an expressed sequence. The data comparison module is configured to compare the first gene expression data with the second expression data to determine the level of binding between the oligonucleotide fragments and the first sample population and between the oligonucleotide fragments and the second sample population. The data analysis module is configured to calculate the genetic differences between the first sample and the second sample by determining which of the oligonucleotide fragments differentially bound to the first and second samples.

In another embodiment the invention comprises a method for determining sequence variations using expression arrays wherein the expression arrays comprise a plurality of oligonucleotide fragments of one or more expressed sequences. The method further comprises the steps of: (a) Analyzing a first sample population using a first expression array to produce a first binding pattern; (b) Analyzing a second sample population using a second expression array to produce a second binding pattern; and (c) Identifying differences between the first binding pattern and the second binding pattern to determine binding differences between the oligonucleotide fragments and the first sample population and between the oligonucleotide fragments and the second sample population.

In still another embodiment the invention comprises a method for identifying sequence variations between a first and second sample population using gene expression arrays. The method further comprises the steps of: (a) Interacting the first sample population with a first expression array to generate a first binding pattern; (b) Interacting the second sample population with a second expression array to generate a second binding pattern; (c) Scaling the first and second binding patterns with respect to one another to create a first and second normalized binding pattern; and (d) Identifying differences between the first and the second normalized binding patterns indicative of sequence variations between the first and the second sample populations.

In a further embodiment the invention comprises a method for identifying sequence variations between a first and second nucleotide sequence using binding information obtained from oligonucleotide array analysis. The method further comprises the steps of: (a) Obtaining binding information from oligonucleotide array analysis using at least two samples wherein a first sample contains a first expressed nucleotide sequence having a first binding pattern and a second sample contains a second expressed nucleotide sequence having a second binding pattern; (b) Normalizing the binding patterns to produce a first scaled binding pattern and a second scaled binding pattern; (c) Comparing the first scaled binding pattern and the second scaled binding pattern to identify differences between the scaled binding patterns; and (d) Associating the differences with sequence variations between the first and the second expressed nucleotide sequence.

In a still further embodiment the invention comprises a method for identifying sequence variations using expression pattern differences. The method further comprises the steps of: (a) Identifying a first expression pattern for a first nucleotide strand and a second expression pattern for a second nucleotide strand wherein the first and second expression patterns are obtained by annealing the first and second nucleotide strands with a complimentary probe set; (b) Comparing the first expression pattern and the second expression pattern and identifying differences between the binding of the first nucleotide strand with the complimentary probe set and the binding of the second nucleotide strand with the complimentary probe set; and (c) Identifying sequence variations between the first nucleotide strand and the second nucleotide strand based upon the binding differences.

### BRIEF DESCRIPTION OF THE DRAWINGS

These and other aspects, advantages, and novel features of the invention will become apparent upon reading the following detailed description and upon reference to the accompanying drawings. In the drawings, same elements have the same reference numerals in which:

FIG. 1 illustrates a high level block diagram of one embodiment of a system for sequence diversity determination.

FIG. 2A illustrates an exemplary oligonucleotide array used in connection with the sequence diversity determination system.

FIG. 2B illustrates hybridization patterns for an analogous expressed sequence lacking sequence diversity.

FIG. 2C illustrates hybridization patterns for an analogous expressed sequence displaying sequence diversity.

FIG. 3 is a flow diagram illustrating one embodiment of a process for performing sequence diversity analysis using array data acquired from two or more sample populations.

FIG. 4 illustrates an overview of the use of array data by the sequence diversity determination processes.

FIGS. 5A, 5B and 5C are line graphs that illustrate the use of hybridization data to identify sequence diversity.

FIGS. 6A and 6B are line graphs that illustrate the use of average difference values in identifying regions of sequence diversity within a nucleotide sequence.

FIG. 7 illustrates a performance profile for a control data set used to identify sequence diversity through D-call identification.

FIG. 8 is a graph illustrating the relationship between expected true positive rates (TPR) and selected D-call values.

FIGS. 9A-C illustrates experimental data obtained from two mouse strains used to identify regions of sequence diversity within selected genes.

### DETAILED DESCRIPTION

Embodiments of the invention include systems and methods for determining sequence diversity between two or more expressed RNA or message populations. Sequence diversity may be characterized by variations in the base composition of an expressed RNA and the corresponding DNA strand that codes for a particular protein or molecule present in an organism or biological sample. Using the methods described herein, mutations and polymorphisms can be identified by analyzing gene expression information to provide additional valuable information not previously obtainable by conventional methods.

One particular feature of the invention provides the ability to utilize gene expression information derived from oligonucleotide arrays and messenger RNA (mRNA) templates to screen for genetic diversity and polymorphisms of sample populations. The disclosed methodology is distinguished from other polymorphism screening methods in that a genomic DNA template is not required. Instead the present methodology makes use of expressed message information (typically derived from labeled RNA or cDNA constructed from mRNA templates) and uses this information to perform polymorphic or sequence diversity analysis.

The expressed RNA or message populations used in the comparison comprise one or more nucleotide strands, genes, or messages that are desirably compared to other populations having similar compositions. Sequence differences are identified using nucleotide-specific hybridization information contained in the raw data generated by expression analysis. In one aspect, sequence differences are identified as changes in one or more bases within the gene, message, or nucleotide strand of interest. For example, if a first message has a sequence composition of “AGTGAATA” and a second analogous message coding for the same or similar protein has a sequence composition of “ATTGAATA,” the methods described herein can identify the single base difference between the two messages. Thus the system and methods described herein provide a means for utilizing gene expression information in a previously undisclosed manner to identify polymorphisms. Use of this analytical technique advantageously increases the amount of useful information that can be obtained from a gene expression experiment. Additionally, the overall cost of performing array-based analysis, is substantially reduced as it is no longer necessary to perform separate polymorphic analysis and gene expression analysis. The system and methods described herein further offer a number of advantages over other conventional methods which are typically laborious, time-consuming and expensive processes. In particular, large numbers of expressed sequences can be rapidly screened for sequence differences in a global manner with little additional effort.

In one aspect, the sequence difference analysis is performed using a gene expression analysis platform that comprises a microarray. The microarray contains a large number of oligonucleotide probes that are used to analyze complex expressed message populations simultaneously. Based on patterns of hybridization formed between the nucleotide probes of the microarray and the nucleotide targets of the expressed message population, individual species may be identified. Furthermore, as will be described in greater detail hereinbelow, the composition of the microarray is such that knowledge of the hybridization properties for specific sequences for each message target can be identified. Differences in hybridization for specific message targets of two or more samples can then be compared to determine hybridization differences that identify potential regions of sequence diversity.

Using the sequence diversity determination methods described herein provides a novel methodology for utilizing data obtained from microarrays and other hybridization-based expression analysis techniques whose use originally was intended to evaluate expression differences between samples. Using the microarrays in this manner provides additional valuable information about sequence differences, mutations, or polymorphisms for large numbers of genes. Furthermore, these methods may be applied to existing data sets from previously performed experiments to look for polymorphisms in the samples without the need for additional sample collection or experimentation.

One embodiment of the invention relates to systems and methods for determining the polymorphic diversity using complimentarily-based approaches to detect differences between one or more expressed sequences. These methods are particularly well suited for analyzing various message populations. Additionally, genetic diversity of analogous expressed sequences in highly complex sample populations, such as those found in whole cell or tissue extracts, can be readily assessed. In the analysis of complex sample populations, array-based gene detection technologies can be used in conjunction with the sequence diversity determination methods to provide a rapid and accurate assessment of sequence diversity arising from polymorphisms in analogous expressed mRNAs, genes or nucleotide sequences present between two or more samples.

Sequence diversity may be determined by comparing the hybridization patterns for a first expressed nucleotide sequence and a second expressed nucleotide sequence to partially homologous or overlapping probe sets bound to an oligonucelotide array. The first and the second expressed sequences may be representative of DNA, cDNA, RNA, cRNA or other nucleotide species that are derived from one or more sample populations and are compared to one another to determine sequence similarities and differences.

In one example, the array desirably contains a plurality of unique fragments that are at least partially homologous to one or more messages of interest. A labeled sample population is hybridized to the array and a first pattern of positive locations on the array along with their intensity level is determined. Additionally, a second labeled sample population is placed on a second duplicate array where each location corresponding to an analogous message is illuminated to the same or similar intensity (after compensating for differences in abundance). Any nucleotide difference between a message in the first sample, and a message in the second sample can lead to a detectable difference in the hybridization patterns on the two arrays. For example, in a first sample, locations 1, 5, 10 and 16 on an oligonucleotide array, each corresponding to a unique probe from a target message might be illuminated. However, a second duplicate array probed with a second labeled sample, may only illuminate locations 1, 10 and 16. These data infer that the target message is likely to contain a polymorphism in a portion of the target gene associated with location 5, since the gene in the second sample did not bind to location 5.

While the above example of polymorphic analysis is described in conjunction with two or more duplicate arrays, it will be appreciated that a similar analysis can be performed using a single array so configured to be able to detect and resolve analogous genes from each sample population independently from one another. One way of resolving two or more sample populations is to differentially label each of the samples.

The invention may be advantageously applied to previously collected gene expression data which can be reanalyzed to identify potential polymorphisms through comparisons of various sample populations. This feature is significant in that genetic differences or sequence diversity between analogous sequences present in complex samples can be readily determined without gathering additional experimental data. Furthermore, the use of expression data in identifying sequence diversity among sample populations represents a novel approach to interpretation of the data. This methodology can be applied to previously collected or archived data and to newly collected data resulting in increased utility and knowledge discovery when performing expression experiments.

In attempting to identify sequence diversity or polymorphisms, a wide variety of sample population comparisons can be made. For example, the sample populations under comparison may be derived from the same organism using similar or different tissues or organs. Additionally, similar or different tissues or organs may be obtained from different organisms of the same species. Furthermore, cross-species comparisons can be made to identify sequence differences between conserved genes, messages, and/or proteins.

In one embodiment, the system and methods for genetic difference determination are adapted to operate with conventional oligonucleotide arrays used for gene expression analysis such as GeneChip® arrays produced by Affymetrix, Inc. (Santa Clara, Calif.). One feature of the genetic difference determination method is that no modification of the oligonucleotide array is necessarily required. Instead, the raw data and information generated using the oligonucleotide array can be processed after collection, thus improving the usefulness of the system. Furthermore, the aforementioned analysis can be used in conjunction with preexisting raw data to generate new information and insights related to sequence diversity that were not previously identified due to a lack of available analytical methods.

While the system and methods presented herein are described for use with oligonucleotide arrays, it will be appreciated by one of skill in the art that the invention may be readily adapted for use with other gene expression/nucleotide detection technologies. It is anticipated that the invention can be used to identify sequence diversity in conjunction with virtually any expression identification technology which contains nucleotide-specific hybridization information and can be collected and compared for one or more sample populations.

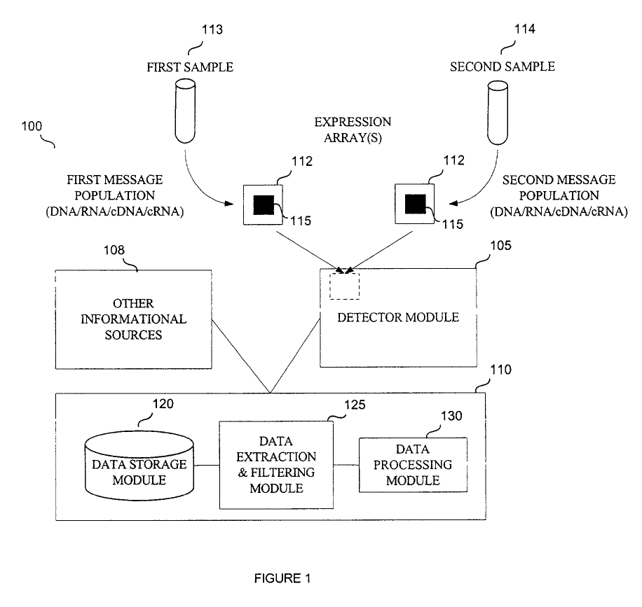

FIG. 1 illustrates one embodiment of a system 100 for genetic difference determination that incorporates an array-based detection approach for analyzing and comparing expression data to determine polymorphic differences between analogous sequences. The system 100 comprises a detector module 105 and an analysis module 110. The detector module 105 processes one or more arrays 112 and resolves/identifies expressed sequences associated with a first sample population 113 and a second sample population 114.

In one aspect, the first and second sample populations 113, 114 are processed by collecting hybridization or intensity information indicative of the coupling or hybridization between expressed nucleotide sequences of the sample populations 113, 114 and one or more probes 115 present in the array 112. The hybridization or intensity information discretely identifies individual expressed nucleotide sequences of the sample populations 113, 114 by detecting specific interactions between the nucleotide sequences in the sample populations 113, 114 and the probes 115 which comprise at least partially complementary sequences with the expressed nucleotide sequences. In one aspect, the coupling between each probe 115 and the complementary expressed sequence in the sample population 113, 114 is quantitated by acquiring signal or intensity information which is subsequently resolved to determine the identity of each expressed sequence as well as identify a relative abundance of the expressed sequence in the sample population 113, 114.

The genetic diversity system 100 uses the coupling information acquired by the detector module 105 and transmits this information to the analysis module 110 for further processing. In one embodiment, the system 100 is adapted for use in performing data processing of array information to provide simultaneously not only expression information but also information describing the sequence diversity between the samples 113, 114. Alternatively, raw data corresponding to expression information from previously processed arrays or other sources of suitable coupling information 108 may be transmitted to the analysis module 110 to determine potential polymorphic differences between the samples. Using the methods described below, valuable information relating to genetic differences between each sample can be extracted from these pre-existing sources of data to generate additional insight into the sequence diversity between the sample populations.

Information obtained from expression experiments that is to be analyzed by the system for sequence diversity determination 100 is passed to the analysis module 110 which receives the information transmitted by the detector module 105 or other informational sources 108. The analysis module 110 performs a genetic diversity analysis to determine the similarities and differences in analogous expressed nucleotide sequences present in the first and the second sample populations 113, 114. In this embodiment, the analysis module 110 comprises a plurality of submodules, including a data storage module 120, a data extraction module 125, and a data processing module 130. The data storage module 120 archives the information transmitted to the analysis module 110 by the detector module 105 and/or other informational sources 108 to permit the information to be stored, retrieved, and updated as necessary to perform gene diversity operations associated with the analysis module 110. In one aspect, the data storage module 120 is configured to store the information in a relational database which can be subsequently accessed to provide discrete information relating one or more of the individual species or expressed messages of the sample populations 113, 114.

In order to determine the genetic diversity of nucleotide species present in the sample populations 113, 114, the data extraction module 125 accesses the information contained in the data storage module 120 and retrieves information relating to one or more specific species of interest that are expressed in the sample populations 113, 114. The data extraction module 120 then associates analogous nucleotide sequence information between the two sample populations 113, 114 in preparation for analyzing the genetic diversity between each desired nucleotide sequence in the sample populations 113, 114.

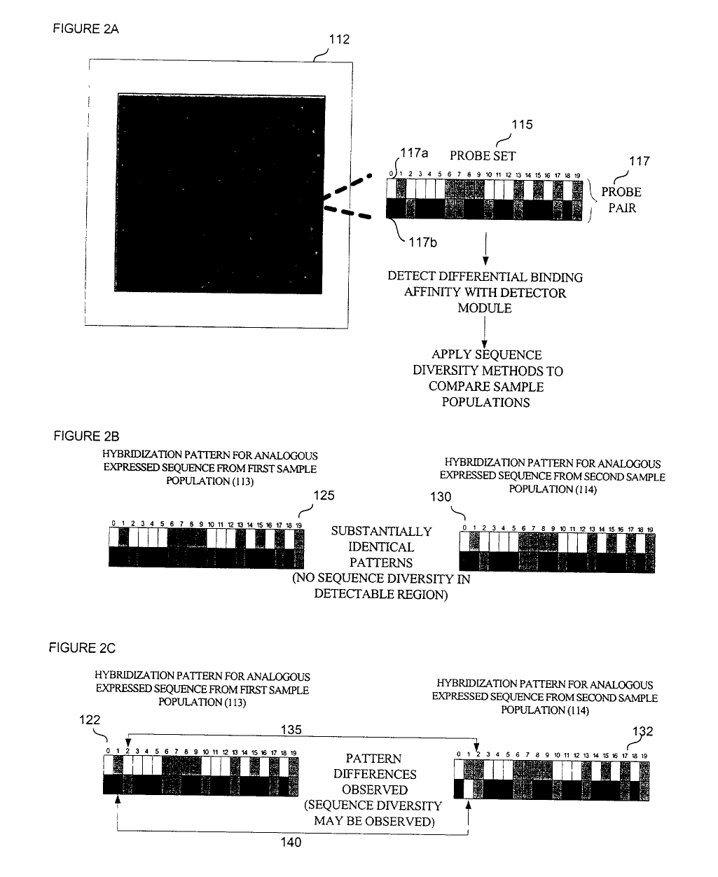

Nucleotide sequence information that has been retrieved by the data extraction module 120 is subsequently received by the data processing module 130 where the information is subjected to genetic diversity analysis to determine the differences between analogous species of the first and the second populations 113, 114. Details of the methodology for performing the diversity analysis will be described in greater detail hereinbelow in connection with FIGS. 2-6. Upon completion of the diversity analysis, the data processing module 130 may update the contents of the data storage module 120 to reflect the analysis results and provide access to the data for subsequent processing.

The system 100 provides functionality for flexibly retrieving the results of the genetic analysis and can be configured to prepare reports, graphs, charts, images, or other data presentations that are used to assess the results of the data analysis. Furthermore, the system 100 may provide means for inputting specific search or analysis queries or retrieving selected results. Additionally, the system 100 operates independently and performs automated data analysis to reduce the requirement for user interaction with the system 100. The automated data analysis may further generate genetic analysis results according to predefined criteria that may be stored and output as requested by a user of the system 100.

As described above, the system 100 comprises a plurality of modules which convey functionality to the system 100. As can be appreciated by one of skill in the art, each of the modules may further comprise both hardware and/or software components that implement various sub-routines, procedures, definitional statements and/or macros used to provide necessary functionality. The modules can also comprise software instructions that are stored to a memory, such as Random Access Memory, Read Only Memory, Electrically Erasable Programmable Read Only Memory (EEPROM) or other similar memory types. The aforementioned description of each of the modules is therefore used for convenience to describe the functionality of the preferred system. Thus, the processes that are undergone by each of the modules may be arbitrarily redistributed to one of the other modules or combined together in a single module.

The system 100 is preferably run on a computer system, such as a personal computer or workstation, that directs the activities of the various modules 105, 120, 125, 130 of the system 100. The computer includes a microprocessor such as a conventional general purpose single- or multi-chip microprocessor including for example, an Intel Pentium® processor, a 8051 processor, a MIPS® processor, a Power PC® processor, or an ALPHA® processor. The microprocessor typically has conventional address lines, conventional data lines, and one or more conventional control lines. As will be recognized by one of skill in the art, the controller and microprocessor may exchange signals and information with the modules 105, 120, 125, 130 and additionally provide access to the other informational sources 108.

In one aspect the system is based on the Microsoft Windows® platform, however, other operating systems such as: UNIX, LINUX, Disk Operating System (DOS), OS/2, MacOS and so forth would also function similarly. Furthermore, any of a number of programming languages such as Perl, SQL, MySQL, XML, C, C++, Java, BASIC, Pascal, and FORTRAN may be used to define functions which implement the analytical methods for sequence diversity analysis.

FIG. 2 illustrates an exemplary oligonucleotide array 112 that is used in connection with the sequence diversity determination system and methods. The oligonucleotide array 112 includes a plurality of probe sets 115 that are present within the array 112 and exposed to the sample populations 113, 114 to provide a means for identifying the expressed species within each population 113, 114. In the illustrated embodiment, each probe set 115 comprises twenty probe pairs 117. The probe pairs 117 further comprise two probe species 117a, 117b whose base sequence is substantially the same as one another but differ by at least one base within their respective sequence. The base composition and probe length for each probe species 117a, 117b is typically known and the probe sets 115 are designed to enable multiple expressed species to be detected at once. In the illustrated embodiment, the length of the probe species used in the array 112 is between approximately 15-60 bases for each probe species. Furthermore, the total number of probe sets 115 contained in the array 112 may be widely varied from as few as one to more than twenty thousand discrete probe sets 115.

In one aspect, the probe pairs 117 that make up each probe set 115 are frequently designed to be partially overlapping such that a portion of each probe species 117a (section 0) and 117a (section 1) may contain an overlapping sequence region that is directed towards substantially the same portion of the target expressed sequence of interest in the sample populations 113, 114 and a non-overlapping region that are directed towards different regions of the same expressed sequence. It will be appreciated that other probe species sections (illustrated as sections 0-19) may possess similar characteristics of sequence overlap in a manner analogous to the probe species 117a section 0 and section 1. Likewise, probe species 117b may comprise similar sections of overlapping sequence corresponding to that of the probe species 117a described above.

As previously described, the probe species 117a, 117b are arranged in pairs and are subdivided into a homologous probe (HM) 117a and a partially homologous probe (PHM) 117b. In one aspect, the HM probe 117a includes a base sequence that is at least partially complementary to a nucleotide sequence in the sample populations 113, 114. The PHM probe 117b also has a base sequence that is at least partially complementary to the same nucleotide sequence however, the base sequence of the HM probe differs from that of the PHM probe 117b by at least one base such that a difference in complimentarity exists between the HM probe 117a and the nucleotide sequence and the PHM probe 117b and the nucleotide sequence.

During expression analysis using the array 112, the nucleotide sequence of the sample populations 113, 114 that has a complementary sequence to the probe set 115 binds to each probe species 117a, 117b within probe set 115 with a different affinity based on the sequence differences between the HM and PHM probes 117a, 117b. This affinity difference is subsequently identified by the detector module 105 (FIG. 1). In one aspect, the differential hybridization affinity is observed as a difference in signal intensity produced as a result of binding between the expressed sequence of interest and the HM and PHM probes 117a, 117b. The analysis module 110 identifies this difference in coupling and stores information that describes the affinity of a sequence of interest to the probes 117a, 117b in the probe set 115. Coupling data is obtained in this manner for the two sample populations 113, 114 and the data is processed using the analysis methods described hereinbelow to identify sequence differences between analogous nucleotide species present in the two sample populations 113, 114.

In one embodiment, the expression analysis may be conducted using a single probe species rather than a HM and PHM probe pair. Using an analogous approach as described above for identifying differential hybridization affinities produced as a result of binding between the expressed sequence of interest and the HM and PHM probes 117a, 117b; the analysis may be conducted using probe species representative of only HM probes. For example, sequence diversity may be identified between two sample populations based on differential binding between a sequence of interest and a corresponding probe species without the requirement for comparison against an additional probe species. It is therefore conceived that sequence diversity can be identified not only by using array configurations in which HM and corresponding PHM probes are formed, but also in other hybridization analysis platforms, arrays, and data sets comprising unpaired probe species (i.e. only HM probes). Additional details of this manner of analysis are described with reference to FIG. 2C below.

One distinction between the sequence diversity identification methods described herein and traditional gene expression analysis methods is that conventional methods fail to identify sequence differences between analogous nucleotide sequences. Instead, conventional methods are concerned with identifying gene expression differences by evaluating and combining results from all probe pairs within the probe set 115. Typically, the combined results are generated using averaging methods designed to discard, ignore, or minimize observed discrete probe pair species differences in favor of producing intensity values and quantified measurements directed toward the complete probe set 115. Sequence diversity determination, on the other hand, utilizes observed coupling differences between sample populations 113, 114 and more specifically between individual probes 117a, 117b in the probe sets 115 to identify regions of expressed sequences that may be divergent between the two or more sample populations 113, 114. Furthermore, the sequence diversity determination methods employ specialized operations that are designed to scale or normalize the coupling data to account for differences in gene expression which may obfuscate the identification of polymorphisms or other alterations in the nucleotide sequence. The present invention is also distinguished from other conventional methods used to screen for polymorphisms in that genomic DNA is not used as a template for analysis. Instead, the sequence diversity determination methods are directed towards manipulating gene expression information (obtained from mRNA or expressed message templates) in a novel manner to extract information that may be used to isolate regions within the expressed messages which may exhibit polymorphic behavior. It will be appreciated that this approach to analysis is an improvement over both conventional gene expression and polymorphism detection techniques as it provides a means for generating comparable information from a single expression data set.

FIG. 2B illustrates an idealized hybridization pattern for an exemplary expressed sequence that lacks sequence diversity between samples 113 and 114 in the region detectable by the probe set 115. In the illustration, each probe set 115 is represented by a hybridization pattern 125, 130 that has been normalized or scaled to account for differences in gene expression between the first and the second sample population 113, 114. The resulting hybridization patterns 125, 130 suggest that for the illustrated expressed sequence, no observed differences between hybridization patterns exist and, therefore, no sequence divergence is expected between the first and the second sample populations 113, 114.

In contrast, FIG. 2C shows an idealized hybridization pattern representative of another exemplary expressed sequence that indicates differences 135, 140 between the hybridization patterns 122, 132 of samples 113 and 114. The first observed difference 135 is shown as a difference between a single HM probe species 117a when compared between the two sample populations 113, 114. The second observed difference 140 is shown as a difference between a single PHM probe species 117b when compared between the two sample populations 113, 114. The analytical methodologies presented herein may be used to identify sequence diversity between one or more HM or PHM probe species 117a, 117b with or without regard for the corresponding probe species when compared between the two sample populations 113, 114. Alternatively, sequence diversity can be identified between corresponding HM and PHM probe species 117a, 117b. In one aspect, the determination of the appropriate probe species 117a, 117b and component sections to be used in the analysis may be selected based on numerous criteria including sequence composition, known or desired homologies, hybridization intensity and other factors used to select one or more portions of the hybridization pattern which are suitable for polymorphic analysis. In each case, the observed difference (exemplified by intensity differences 135, 140) may be indicative of differences in hybridization affinity arising from a sequence polymorphism in the two sample populations 113, 114. The methods presented herein identify these regions of potential sequence diversity and localize the regions in the expressed sequences where polymorphisms may exist.

Briefly stated, sequence diversity is identified as a result of observing hybridization differences between each sample population 113, 114 that give rise to alterations in hybridization efficiencies when comparing the hybridization profiles for the sample populations 113, 114. It will be appreciated that although the following description describes the identification of sequence diversity within a single expressed sequence, the methods can be readily applied to assess a plurality of expressed sequences present in each sample population 113, 114 to identify differences between several different analogous sequences. In one embodiment, a complete assessment of all probe species 117a, 117b contained in the array 112 can be performed resulting in the assessment of sequence diversity for many thousands of messages or expressed sequences.

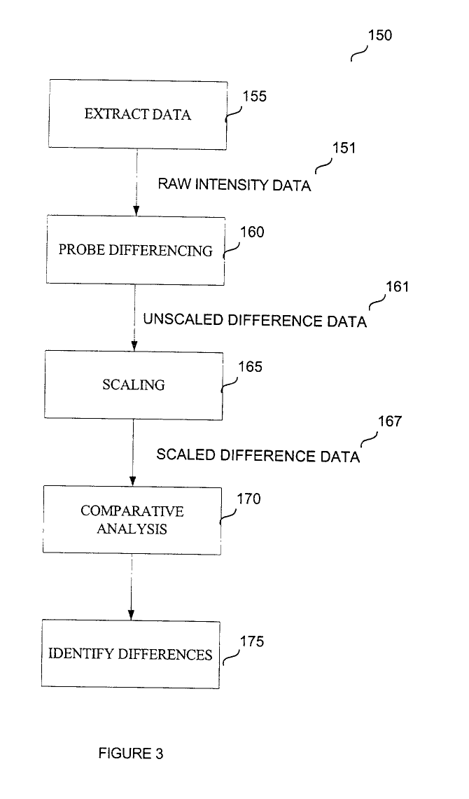

FIG. 3 illustrates one embodiment of a process 150 for performing sequence diversity analysis using array data acquired for two or more sample populations 113, 114. As previously indicated, the array data may comprise expression data collected after coupling between the sample populations 113, 114 and the probe species 117a, 117b of the array 112. The expression data is typically collected in an archival form and comprises raw signal information 151 representative of discrete signal intensities or values obtained for each probe species 117a, 117b in the probe set 115. In one aspect, the raw data 151 is extracted at a state 155 prior to processing by conventional nucleotide array instrumentation. Because the module utilizes the raw gene expression data, this insures that the binding differences are not removed or lost as part of the gene expression analysis process.

The process 150 proceeds by identifying and isolating data that is associated with the expressed sequences of interest at a state 160. Typically, data corresponding to analogous expressed sequences from each sample population 113, 114 are identified and processed by the data processing module 130. In one aspect, the data processing module 130 includes a microprocessor cluster having a plurality of processors that process the data in a parallel or distributed manner to improve the performance or speed of analysis. During data analysis, a direct comparison of signal information from each probe species 117a, 117b of interest from the two or more sample populations 113, 114 is made. In one embodiment, the array intensity information is derived from probe pairs 117 including both the HM probes 117a and PHM probes 117b. For each probe set 115, a preliminary analysis of the intensity values from selected raw data 151 derived from HM probes 117a and the PHM probes 117b is acquired by the system and these values are differenced against one another to generate unscaled intensity values 161 for each probe pair 117.

The unscaled intensity information 161 then undergoes a scaling procedure at a state 165 that internally normalizes the intensity information with respect to the sample population 113, 114 from which the intensity information was obtained. The scaling procedure undertaken at the state 165 may incorporate a replicate analysis procedure where intensity values are averaged across more than one sample derived from the same sample population 113, 114. Averaging in this manner increases the confidence level or predictive quality of the hybridization or intensity information for each sample population when compared to other sample populations. The manner in which the data is scaled and averaged may employ techniques and analytical calculations that are well known in the art for processing discrete intensity or hybridization signals relative to the complete compliment of probes 117a, 117b contained in the array 112.

In one embodiment, during or subsequent to the abovementioned differencing or scaling procedures, an outlier removal function may be used to identify and remove data that exceeds a statistical threshold of reliability. Outlier data is characterized by spurious or inaccurate hybridization/intensity values and may not reflect actual hybridization intensities expected for a particular sequence. Outlier data may arise from experimental error or inaccuracy, systematic errors, localized regions of abnormally high and/or low intensity on the surface of the microarray, and other sources. The analytical methods for sequence diversity determination may desirably incorporate methods to identify outlier data for example by identifying data which exceeds a statistical threshold of reliability. Furthermore, inadequate hybridization signals from samples where a gene is not present are filtered out before analysis to prevent identifying differences due only to a background signal of noise and not to real sequence differences. These data may then be excluded from further analysis to minimize improperly identified sequence differences (e.g., false positives). Additional details of the methods used for determining statistical reliability of the data will be described in greater detail hereinbelow.

After transforming the unscaled data 161 into scaled data 167, each sample population 113, 114 is then compared at a state 170 with respect to one another. Due to expression differences or differences in relative abundance between the sample populations 113, 114, a second normalization or scaling procedure may be performed in the state 170 to the scaled difference data 167. The purpose of the second normalization or scaling procedure is to generate an apparent level of expression or expressed sequence abundance that permits the direct comparison of the scaled data 167 between the sample populations 113, 114. Although this second scaling may improve the comparison of samples prepared differently, it is not necessary to identify sequence differences reliably between two sample populations. If samples have been prepared grossly differently, another step of scaling may be considered. Pattern or scaled data differences in probe pairs 117 or probe sets 115 that are observed are then identified at a state 175 as candidate regions where sequence diversity or polymorphic sequence information may be found. Furthermore, using the known sequence of the probes 117a, 117b, specific regions within the expressed sequence that have been determined to contain sequence diversity can be further identified and the polymorphism localized.

In one aspect, the aforementioned sequence diversity analysis process 150 may include a filtering step which identifies noise-related signals or non-specific hybridization between the sample populations. Noise identification is useful to prevent anomalous analysis of signals or intensity information which may not accurately represent true sequence diversity between the samples. In particular, the filtering step may be used to prevent signals, composed predominately of noise or whose actual sequence hybridization intensities cannot be resolved from the underlying noise, from confounding the identification of sequence diversity between the samples. The filtering step can further be used to identify and remove noise from the hybridization signal such that the resulting filtered signal corresponds substantially to the actual hybridization information from the sample of interest.

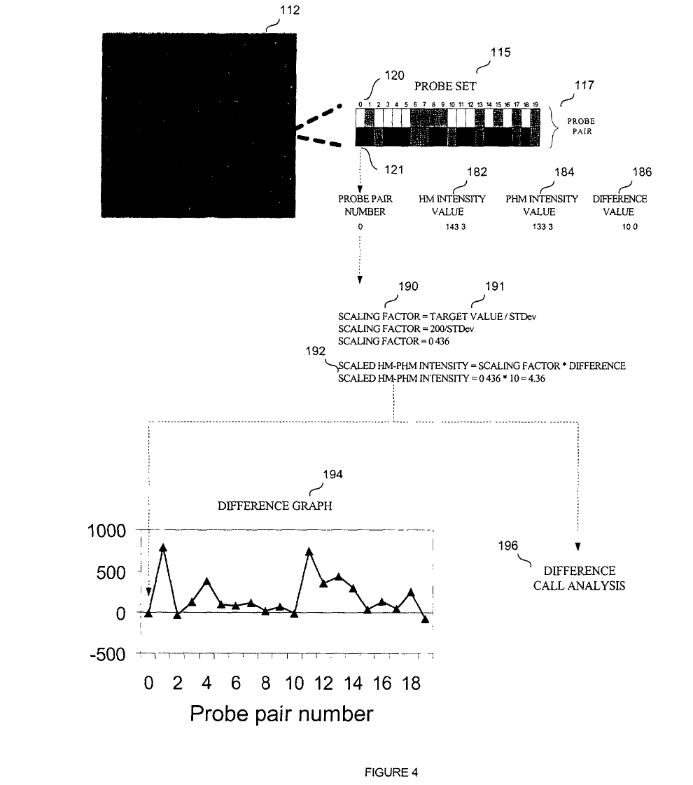

FIG. 4 illustrates one embodiment of a method for processing of oligonucleotide array data for a selected message or expressed sequence using the aforementioned sequence diversity determination method 150. In the illustrated embodiment, expression hybridization or intensity data corresponding to an expressed sequence of interest is extracted from the oligonucleotide array 112. As previously described, the expression data may comprise one or more probe sets 115 that contain information describing the hybridization efficiency between the selected expressed sequence and the probe species 117a, 117b of the probe set 115. For each probe set 115, a plurality of probe pairs 117 are identified that contain hybridization values for the HM probe 117a and the PHM probe 117b across selected regions of the nucleotide sequence. In one embodiment, the oligonucleotide array 112 used in the analysis may comprise an expression array such as the GeneChip® array manufactured by Affymetrix, Inc. and the hybridization or expression data used in the analysis is derived from an intensity file or “.cel” file which contains raw hybridization information and numerical data for each message or expressed species for which the array was designed to detect. The “.cel” intensity file comprises a processed form of the raw data that contains a single intensity value for each probe species 117a, 117b associated with the 75^{th} percentile of pixel intensity distribution and is indicative of the hybridization signal generated by the coupling of each probe species 117a, 117b with the complimentary expressed species.

The analysis continues with each probe pair 117 in the probe set 115 being isolated and a raw difference calculated between the HM probe 117a and the PHM probe 117b. For example, as shown in FIG. 4 for probe pair “0” an HM intensity value 182 of “143.3” is identified and a PHM intensity value 184 of “133.3” is identified. A difference value 186 is then calculated for the probe pair 117 based on the observed intensity values. Thus, the difference value 186 for probe pair “0” is determined by taking the difference of the HM intensity value “143.3” and the PHM intensity value “133.3” to yield a difference value of “10.0”. This raw difference calculation is performed for each probe pair 117 in the probe set 115 to produce a plurality of raw difference values 186, one for each probe pair 117.

After the raw difference values 186 have been determined, scaling analysis is performed to globally scale the raw difference values 186. In one aspect, scaling compensates for inter-region and inter-strain expression differences and allows two or more sample populations 113, 114 to be more accurately compared. As will be appreciated by one of skill in the art, scaling analysis can be performed in a number of ways with each method capable of performing the operations necessary to allow the two or more sample populations 113, 114 to be compared. One desirable form of scaling analysis employs the use of a scaling factor 190 that is determined in part by calculating the standard deviation for the intensity values across each of the probes 117a, 117b in the probe set 117. As shown in the illustration, the scaling factor 190 is determined by dividing a target value 191 by the standard deviation of the intensity differences.

The target value 191 used in the calculation may be empirically determined and is dependent upon the characteristics and type of gene expression or intensity data used in the analysis. In one aspect, the target value 191 is selected based on a desired internally scaled intensity that is desirably used across all probe sets 115 that are compared. Furthermore, the standard deviation calculation used to generate the scaling factor may be trimmed where the highest and lowest intensity values in the probe set 115 are discarded. Discarding the highest and lowest differences when performing the scaling factor calculation alleviates skewing resultant from large positive or negative outliers in the probe set 115. It will be appreciated that the aforementioned description of scaling factor determination represents but a single approach to scaling of the data to account for hybridization differences between the sample populations 113, 114. It is contemplated that other scaling techniques exist and the choice of scaling technique to be used is dependent upon the type, organization, and quality of the data to undergo diversity analysis. Therefore, other scaling techniques used in conjunction with the system and methods described herein are considered to be additional embodiments of the present invention.

After determining the scaling factor 190, each raw difference value 186 is transformed into a scaled difference value 192 by applying the scaling factor 190 to the difference value 186. For example, for probe pair “0” the scaled difference value 192 is determined as the product of the scaling factor 190 and the raw difference value 186.

The aforementioned process is applied for each probe pair 117 to generate a plurality of scaled difference values 192 that are representative of the coupling interactions for the expressed sequence of interest with the probe species 117a, 117b of the array. Taken together, the plurality of scaled difference values 192 can then be used for comparing the two sample populations 113, 114 in a manner that will be described in greater detail hereinbelow.

As shown in FIG. 4, one method by which the data can be visualized is by producing a difference plot or graph 194 that contains each of the scaled difference values 192. The difference graph 194 facilitates visualization of the transformed data and may be helpful in identifying sequence differences between the sample populations. Additionally, as will be subsequently described in greater detail, the scaled difference values 192 may be desirably used in a difference call (D-Call) analysis 196 to determine sequence differences between the sample populations of interest. The difference call analysis 196 determines a D-call value for each probe pair and is used in the comparison of the sample populations 113, 114 to identify pattern differences that are statistically significant and may be representative of sequence differences between the sample populations 113, 114.

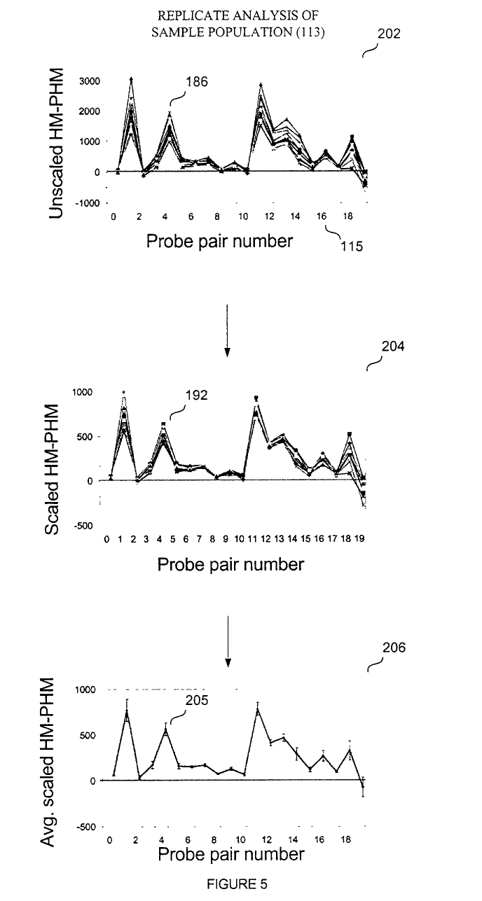

Although not required for graphical analysis, it is often desirable to perform replicate analysis for each sample population 113, 114 to increase confidence in the data analysis, to reduce the effects of spurious or outlier data points and to be able to perform statistical analyses. When performing replicate analysis, scaled difference values 192 representative of replicated data for a single sample population 113, 114 may be averaged to generate an average scaled intensity value 200. Thus as shown in FIG. 5, for a particular probe set 115, a plurality of identical probe pair calculations may be performed that are derived from a plurality of independent samples or data sets collected using the same sample populations or an equivalent sample population that are to be desirably combined to reflect the characteristics of a single sample population 113 or 114. In this illustration, a difference graph 202 is shown for a plurality of unscaled difference values for a single probe set 115. For each probe pair 117, a plurality of unscaled difference values 186 is shown and is representative of replicated data from each of the sample populations 113 or 114 to be combined. As is reflected by the data contained in the graph 202, the range of the unscaled difference values 186 varies from one probe pair to the next. In certain instances, the range of the unscaled difference values 186 demonstrate significant distribution of results as is shown by the distribution of unscaled difference values for probe pairs “1”, “4”, “11”, and “14”. Alternatively, other probe pairs show less variation in the unscaled difference values 186 as is noted for probe pairs “0”, “3”, “5”, “6” and “7”.

The scaling process that is applied to the unscaled difference values 186 results in a “tightening” of the data as shown by the scaled graph 204. For each probe pair 117, the new distribution of replicated scaled difference values 192 indicates the relative range of confidence or error distribution. The error distribution is then consolidated into a single data point by averaging the scaled difference values 192 to generate an averaged scaled difference graph 206 showing a single average difference value 205 for each probe pair 117. Additionally, the average difference value 205 may be accompanied by an error range or error for indicating the confidence level based on the distribution of data from which the average difference value 205 was derived. The average difference values 205 and the associated graph 206 may then be compared to similar information obtained for the second sample population 114 that has undergone a parallel analysis in a similar manner to that described above.

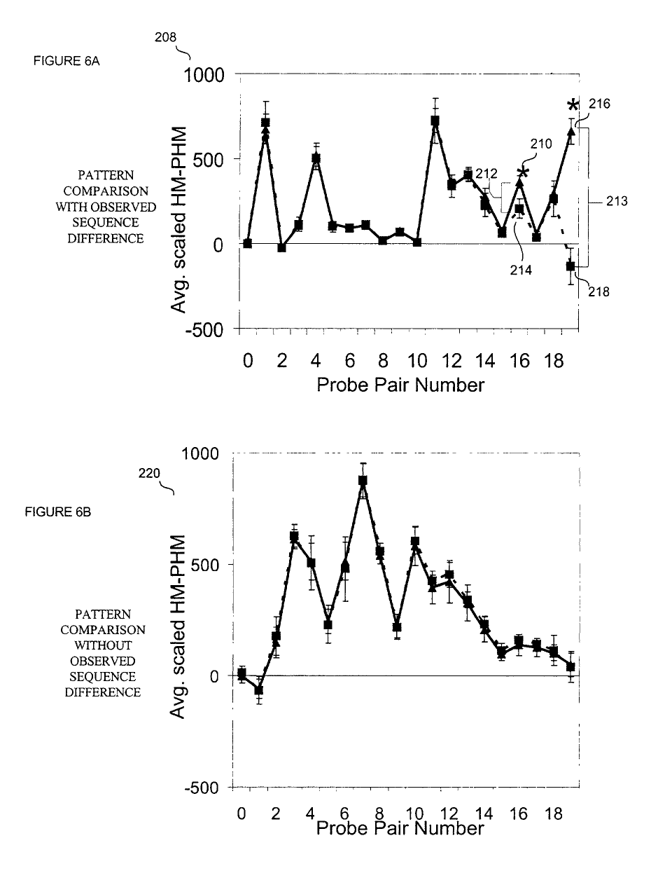

FIGS. 6A and 6B illustrate the use of average difference values 205 and associated graphs 206 in identifying sequence diversity between a first sample population 113 and a second sample population 114. In the illustrated embodiments, averaged scaled difference values 205 derived from hybridization data for a single probe set 115 using a first sample population 113 and a second sample population 114 are shown. For each probe pair 117 in the probe set 115, scaled difference values 205 corresponding to values obtained from the two populations 113, 114 are plotted. The resulting comparison graph 208 is used to identify regions of the nucleotide sequence that may contain sequence diversity based upon observable differences in the hybridization behavior of the expressed sequence with the probe species 117a, 117b. When comparing the composition of the expressed sequence of the first population with the analogous expressed sequence of the second population, it will be expected that the hybridization behavior of identical regions of the expressed sequence which lack any differences in sequence will display similar patterns of intensity (i.e., the exemplary patterns shown in FIG. 2A).

As shown in FIG. 6A, the hybridization characteristics of the expressed sequence and the probe pairs, as determined by the scaled differences values 205, indicate that the composition of the expressed sequence observed in the two sample populations 113, 114 is identical along the majority of probe pairs 115. This observation is based on similarities in scaled difference values obtained at probe pair positions “0-15” and “17-18”. At probe pair position “16”, however, there is a significant difference 212 between the scaled difference value 210 obtained from the first sample population 113 and the scaled difference value 214 obtained from the second sample population 114. Likewise, at probe pair position “19” there is a significant difference 213 between the scaled difference value 216 obtained from the first sample population 114 and the scaled difference value 218 obtained from the second sample population 114. These observed differences exceed the threshold of uncertainty given by the error bars for each scaled difference value and therefore are representative of hybridization differences in the analogous expressed sequence between the two sample populations. These hybridization differences cannot be accounted for by experimental or systematic error alone and suggest the expressed species in the two sample populations may be divergent with respect to their base composition. Significant differences in hybridization between the two sample populations 113, 114 observed for specific probe pairs in this manner may therefore identify potential regions of the expressed sequence that exhibit polymorphic behavior or sequence diversity.

FIG. 6B illustrates a scaled difference value graph 220 for an analogous expressed sequence present in two sample populations 113, 114 that does not exhibit polymorphic behavior in the region detectable by the probe species 117a, 117b of the probe set 115. As described above in connection with FIG. 6A, scaled difference values 205 are plotted for the first sample population 113 and the second sample population 114 for each probe pair 117 of the probe set 115. Any significant differences observed between the scaled difference values 205 between the sample populations 113, 114 for a particular probe pair 117 would be indicated by an observable difference in the scaled difference values 205. In the case of expressed species with non-polymorphic or identical sequences, there are no significant differences between the plot of the first population and the plot of the second population. From this data it can be determined that the expressed sequence of interest contains an identical sequence for each sample population 113, 114 in the region detectable by the probe set 115 and, therefore, does not exhibit any sequence diversity in these regions.

While the aforementioned description of the methods by which a polymorphic sequence is determined incorporates the use of graphs or plots of the data, it will be appreciated that the system is not required to utilize graphical or plotting techniques exclusively. For example, the system 100 can apply comparative routines that compare the hybridization results for the sample populations 113, 114 directly using the numerical values. An identification threshold can further be used such that when the difference between the average scaled value of the first population and the average scaled value of the second population exceeds a threshold value, the system identifies this region of the expressed species as being potentially polymorphic in nature. Furthermore, it will be appreciated that the system and methods described herein may be applied to compare scaled difference values directly rather than requiring the calculation of average difference values of replicated data sets, however this approach may reduce the type of statistical analyses that can then be performed.

In one aspect, putative polymorphisms in the expressed sequence of interest are identified by applying a difference call (D-call) to the scaled difference values 205 to aid in the identification of hybridization differences that may be associated with sequence diversity. The D-call calculation serves as a basis for adjusting the sensitivity of the scaled difference comparison and may be desirably used to reduce the number of false positives reported. Equation 1 illustrates the calculation used to determine the D-call value.

$$\frac{\vert AvgScaledDiff ( {ProbePair}_{A} , {SamplePopulation}_{1} ) - AvgScaledDiff ( {ProbePair}_{A} , {SamplePop}_{2} ) \vert}{( \sqrt{{StdDeviation ( {ProbePair}_{A} , {SamplePopulation}_{1} )}^{2} + {StdDeviation ( {ProbePair}_{A} , {SamplePop}_{2} )}^{2}}} \qquad \text{EQUATION 1}$$

Briefly described, the difference calculation (D-call) applies a mathematical approach to calculating average hybridization pattern differences. More specifically, the D-call is a measurement of the difference in the hybridization pattern at a particular probe pair 115 that can be used to assess data confidence. The D-call calculation is obtained by taking the absolute difference of the average scaled difference values (AvgScaledDiff) for each probe pair in each population. This result is then divided by the square root of the sum of the squares of the scaled standard deviations for each probe pair in each population, yielding the D-call value.

The D-call value is applied by comparing the calculated value with a designated threshold to eliminate outliers that are displaced a fixed number of standard deviations away from the mean of the other values of the probe set 115. For example, a threshold value of “1.8” may be designated and each calculated D-call value for probe pairs 117 within the probe set 115 can be compared against this value. Those calculated D-call values that fall below the threshold value are removed from further consideration as they may present unreliable results which may lead to an increased number of false positive or false negative results.

As an estimate of the false positive rate for a data set, like samples may be compared to each other. For example, using a sample set comprising two or more substantially identical or replicated samples, a portion of the sample set may be compared to another other portion of the same or similar sample set. In one aspect, approximately half of the replicated samples are compared to the other half of the replicated samples. This methodology may be desirably utilized in conjunction with numerous sample types including the C57BL/6J (B6) and 129S6/SvEvTac (129) mice strains described in Example 1 below.

When comparing an isogenic strain to itself (for example B6 to B6 or 129 to 129), no sequence differences are typically expected. Thus an expected false positive rate may be determined when comparing samples with similar or identical genotypes that may be adapted for use with the disclosed sequence diversity analysis methods. In one aspect, estimation of the false positive rate in this manner is useful for assessing sequence diversity across large subsets of candidate genes or sequences and may be conveniently integrated into whole chip analysis. The D-call threshold can then be selected to yield a minimal number of false positives.

In another aspect, the analysis methodology is sensitive to the number of samples analyzed. Using an increased number of replicate samples may increase the reliability of the measured data and therefore reduce the observed rate of false positives. Estimation of the false positive rate further provides useful information when determining the D-call threshold for a specific data set.

In one aspect, the D-call value is used in conjunction with replicate data sets and a sequential calculation process is applied to refine the D-call after eliminating outlier data. In this process, the average and standard deviations of the scaled probe pair differences for the replicated data are calculated and the highest and lowest probe pair differences are eliminated from the data set. Additionally, any probe pair differences that exist a fixed number of standard deviations away from the original average may also be eliminated. The averages and standard deviations for the remaining probe pairs are then re-calculated and the D-call value is calculated as before using the reduced data set. This manner of progressive D-call calculation may improve the identification of true regions of sequence diversity and may reduce improper identification of false positive or false negative results.

It will be appreciated that data confidence can be assessed by a number of different methods. For example, the D-call calculation can be used in conjunction with the student T-test, Wilcoxon signed rank test, Fischer test, ANOVA or other similar statistical methods of analysis to determine data confidence for each probe pair intensity measurement. Therefore, the use of alternative statistical methods in assessing the data confidence and identifying sequence diversity using the methods described herein in place of the D-call calculation are contemplated to be but other embodiments of the present invention and may be used concomitantly with the D-call calculation to determine more accurately the appropriate threshold levels.

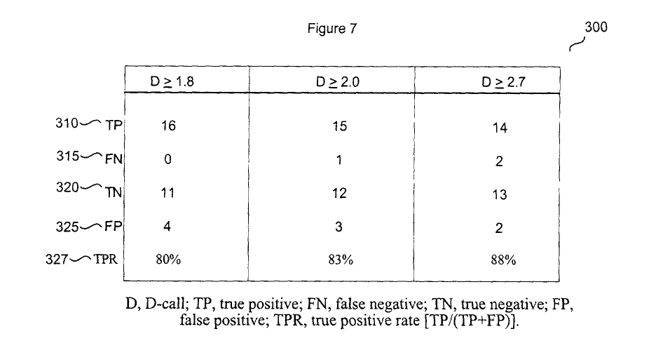

FIG. 7 illustrates an exemplary performance profile 300 for a control data set including nucleotide sequences previously determined to exhibit known polymorphisms. The results of the application of the D-call calculation are applied to three different threshold values 305 including; “1.8”, “2.0”, “2.7”. The profile 300 further summarizes the results based on the accuracy of the call made. The calls include: true positives (TP) 310 representative of expressed sequences having known polymorphisms within their sequence that were properly identified; false negatives (FN) 315 representative of expressed sequences having known polymorphisms within their sequence that were not identified; true negatives (TN) 320 representative of expressed sequences lacking polymorphisms within their sequence and properly identified as such; and false positives (FP) 325 representative of expressed sequences lacking polymorphisms within their sequence but identified as containing a polymorphism. As indicated by the values present in the “1.8” threshold column, an increased number of false positives 325 are identified relative to the “2.0” and “2.7 thresholds. Similarly, as is shown in the “2.7” threshold column, increased number of false negatives 315 were generated relative to the “1.8” and “2.0” threshold values. In summary, the threshold level is determined by the preference of the user for either minimizing false negatives or minimizing false positives. In one aspect, a value representative of the true positive rate (TPR) 327 is determined and is based on the calculation: TP/(TP+FP). This value 327 may be used as a metric for determining the desired D-call stringency to be used during the sequence diversity analysis.

The results of the performance profile 300 indicate the utility in identifying the D-call value and comparing against the designated threshold values. By conducting experiments using control sequences, an “optimized” threshold value can be obtained and this value can be applied to the determination of which D-call values and associated data sets should be excluded from the analysis of sequence diversity to reduce false or misleading results.

Furthermore, as an estimate of the false positive rate for a data set, like samples may be compared to each other, for example, comparing half of the samples to the other half of the samples. As previously described, when comparing one isogenic strain to itself, no sequence differences are typically expected. In one aspect, the analysis methodology is sensitive to the number of samples analyzed and the false positive rate may be estimated with increased frequency when the number of files used in the replicated sample comparison is small. Therefore, even when comparing a relatively small number of replicated samples, a high observed false positive rate is informative when determining the D-call threshold for a specific set of data.

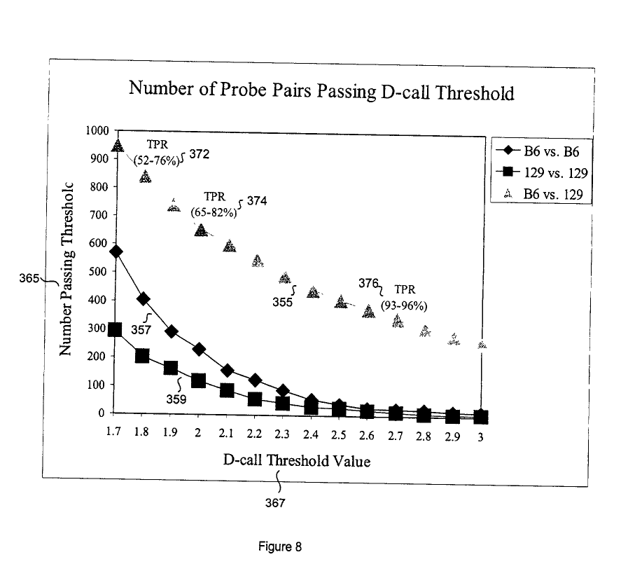

FIG. 8 further illustrates the relationship between expected true positive rates (TPR) and selected D-call threshold values when assessing probe pair hybridizations arising from two mouse strains (B6 and 129) for all probe pairs on Affymetrix Mu11k SubA and SubB chips, comprising 13,069 probe sets (approximately 260,000 probe pairs). Threshold profiles 355, 357, 359 corresponding to a B6 vs. 129 comparison, a B6 vs. B6 comparison, and a 129 vs. 129 comparison are shown respectively. The data point values along each threshold profile 355, 357, 359 further indicate the number of probe pairs 365 exceeding the selected threshold value 367 when comparing hybridization data obtained from selected probe pair hybridization sets for the two mouse strains. The TPR estimated using the intra-strain comparisons is a low approximation because the number of false positives is sensitive to the number of samples compared. For example, twelve samples of B6 are compared to twelve samples of 129 for the interstrain comparison, whereas only six 129 samples are compared to six 129 samples and six B6 samples are compared to six B6 samples. As shown in the illustration, using a D-call value of 1.8 the lowest expected TPR 372 is approximately 52-76%. Similarly, for a selected D-call value of 2.0 the lowest expected TPR 374 is approximately 65-82%. Finally, for a selected D-call value of 2.7 the lowest expected TPR 376 is approximately 93-96%.

The exemplary comparisons demonstrate that as the D-call value increases, the expected TPR likewise increases. Thus, the stringency of the analysis may be readily modified to result in a desired TPR range. This aspect of the invention is significant as it allows the user to analyze data with a high degree of flexibility. In one aspect, the user may select a lower D-call threshold to obtain an increased number of candidate regions where sequence diversity may exist between samples. Alternatively, the D-call threshold may be upwardly adjusted to increase the probability that the identified candidate regions will reflect true sequence diversity.

It will be appreciated that the aforementioned exemplary comparisons and threshold profiles 355, 357, 359 are but one example of the type of analysis that may be conducted to characterize candidate regions of sequence diversity. It is conceived that the actual D-call values used in a given analysis may differ from these values. Furthermore, the D-call value 367 may be selected by the user to accommodate a desirable balance between the quantity of identified regions of candidate sequence diversity and the frequency of false positive and/or negative results.

The system and methods described herein indicate that the analysis of hybridization patterns obtained from gene expression measurements using oligonucleotide arrays can be useful in identifying genes or expressed sequences that contain genetic differences. Furthermore, these genetic differences may be responsible for interesting phenotypes and may aid in the identification of polymorphic sequences related to disease states. In one aspect, the analysis for sequence diversity is highly flexible and can be used in conjunction with numerous types of data obtained from expression studies using microarrays, oligonucleotide arrays, or other technologies.

One feature of the methods for determining sequence diversity is that they may be applied to previously existing data, initially obtained for other purposes, to search in a broad and unbiased manner for genetic differences without the need for any additional experiments. Furthermore, the sequence differences detectable by these approaches can identify small sequence differences (e.g., single-base substitutions), larger sequence differences (e.g., large deletions), and genes or expressed sequences with different splice forms or variants. Thus, these methods can be used to complement not only existing expression studies but also more traditional studies to accelerate the identification of genes or messages that contain sequence diversity that may mediate important phenotypes.

It is conceived that the system and methods described herein may be applied to both inter-species and extra-species comparisons for determination of sequence diversity. For example, expressed sequence diversity may be determined between samples derived from different tissues or cells from the same organism. Alternatively, sequence diversity can be assessed between different organisms within the same species or between different species entirely. In one aspect, sequence diversity can be assessed in biological samples including but not limited to: animals, plants, bacteria, viruses, and fungi. Additionally, the methods described herein may be desirably applied to the assessment of expression information derived from humans, rats, mice, drosophila and other organisms for which genetically sufficient information is available.

In one aspect, the system and methods described herein may be used to identify and evaluate disease states related to polymorphic or mutational genetics. For example, polymorphisms between diseased individuals and healthy individuals may be screened by comparing biological samples from selected individuals having a particular disease to biological samples corresponding to normal or healthy individuals. The ability to screen many thousands of genes or expressed sequences simultaneously, representative of the partial or total genetic makeup of the individuals can be useful in associating particular polymorphisms or mutations with a disease condition. This information can further be used in conjunction with expression data obtained from the microarray to provide a more detailed genetic profile to compare samples from normal and diseased individuals. As such, these methods can be desirably used to screen for important polymorphisms and/or mutations that give rise to a particular disease state. As previously described, the sequence diversity analysis can be performed using preexisting microarray data thereby reducing the need to perform new experiments and also providing a useful method for reinterpreting the data.

In another embodiment, evaluation of sequence diversity in the manner described herein may be used to associate polymorphisms or mutations with phenotypic characteristics or traits. For example, cell or tissue samples having one or more distinguishable phenotypic differences may be compared to identify polymorphisms that are linked to a phenotype of interest. Additionally, the specific gene or expressed sequence giving rise to an altered phenotype between cell or tissue samples may be further identified including the localized region within the gene where the polymorphism occurs. Sequence diversity analysis in this manner provides the ability to rapidly screen and assess many thousands of genes at once to identify and isolate potentially significant polymorphisms through the use of differential hybridization analysis.

### EXAMPLE 1

In order to develop and test the analysis methodology, and to find genes with sequence differences, high quality gene expression results were obtained for six different brain regions, in duplicate, for both C57BL/6J (B6) and 129S6/SvEvTac (129) mice. Using this data, the analysis methodology compared the hybridization patterns across 20 oligonucleotide probe pairs per gene, after normalizing for expression level differences, to find probes showing consistent, statistically significant differences between the two sets of samples.

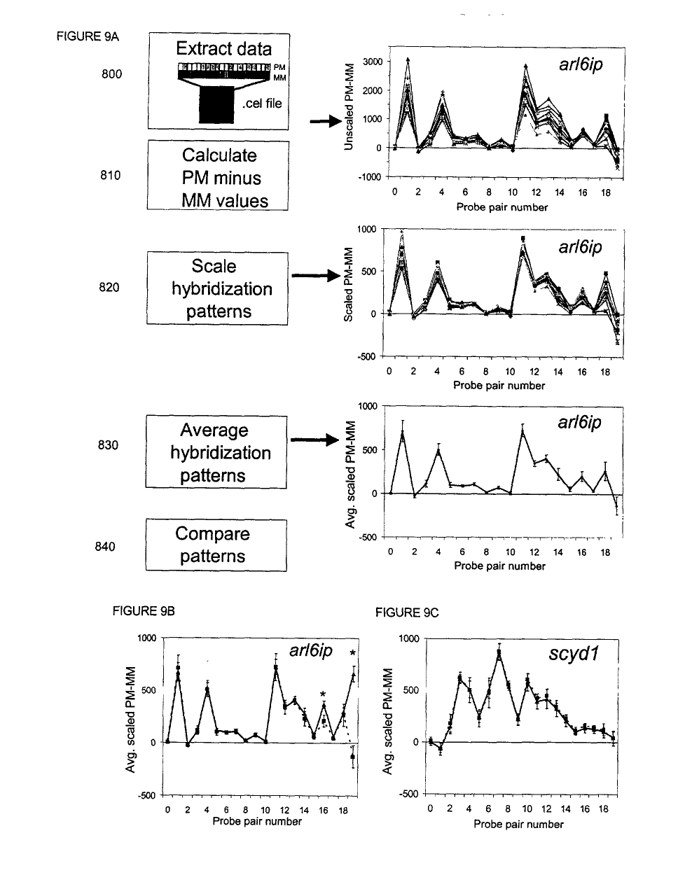

FIGS. 9A-C illustrates the experimental analysis of the aforementioned mouse strains to detect sequence differences using oligonucleotide array expression data. In FIG. 9A, the steps in the analysis method are described (left panels in boxes), and corresponding graphical representations of the data for the gene ADP-ribosylation-like factor 6 interacting protein (arl6ip) are shown on the right. Initially, in state 800, the method extracts data for a specific gene (probe set) from the cell-by-cell intensity (.cel) file. A probe set is comprised of twenty different oligonucleotide probe pairs that hybridize to specific regions of a gene. Each of the probe pairs comprises a matched set of two 25-base oligonucleotide probes further comprising a perfect match (PM) for the gene of interest and a mismatch (MM) containing a single nucleotide substitution in the middle of the probe (position 13) for which the individual hybridization intensity values are extracted. It will be appreciated that the perfect match (PM) probe and the mismatch (MM) probe generally correspond to the aforementioned homologous match (HM) probe and partially homologous match (PHM) probe respectively. The sequence and configuration of the PM and MM probes therefore are representative of but one embodiment of a suitable probe pair that may be used in sequence diversity analysis. In state 810, the hybridization intensity difference between the perfect match and mismatch probe (PM-MM) for each probe pair is calculated for each of the 24 samples (six brain regions, two duplicates, two strains), excluding data sets that did not meet certain pattern quality measures. The unscaled values for arl6ip are shown for all twelve samples of the 129 strain, where the probe pair number is indicated on the x-axis ranging from 0 to 19, and the PM-MM value is shown on the y-axis. Next in state 820, the PM-MM values for each of the probe sets for each sample are globally scaled (by a factor determined from the standard deviation across the multi-probe pattern obtained in each experiment) to compensate for inter-region and inter-strain gene expression differences. The scaling factor is calculated by dividing an arbitrary target value (200 in the example shown) by the standard deviation of the central eighteen PM-MM values for a probe set (i.e., ignoring the largest and smallest of the 20 PM-MM values). To minimize false predictions of sequence differences due to inadequate hybridization signal, the data for a sample may be excluded unless approximately 65% or more of the PM-MM values for the probe set are positive. After ignoring samples that do not fulfill these criteria, the scaled probe pair differences are averaged in state 830 to generate a single value for each probe pair in each strain and scaled again in a similar manner as described above. The scaled averages and standard deviations are shown for arl6ip in the 129 strain. Probe sets where at least four samples for both strains meet the above thresholds are then compared in state 840.

As illustrated in FIG. 9B, for the arl6ip probe set, the same analysis was performed for the twelve B6 samples. The average hybridization patterns with standard deviations obtained for 129 (dashed line and squares) and B6 (line and triangles) mice are shown. At a D-call threshold of 1.8, the algorithm predicts two sequence differences (indicated by asterisks). The “difference call” (D-call) is calculated for each probe pair to identify strain-specific pattern differences that are statistically significant. The D-call approach is found to perform substantially better than a student's t-test and is computed for each probe pair (PP) (see Equation 1). Consistent with the hybridization pattern differences, DNA sequencing identifies two separate single base differences between B6 and 129 in the arl6ip gene within the region covered by the probe set.

As illustrated in FIG. 9C, the average hybridization signals with standard deviations for the small inducible cytokine subfamily D, 1(scyd1) probe set, using data obtained for 129 (dashed line and squares) and B6 (line and triangles) mice, are shown. DNA sequencing identifies no sequence differences between strains, consistent with the nearly identical, overlapping hybridization patterns.

To determine the performance of the algorithm and to optimize the D-call threshold, 27 genes represented by 31 probe sets were randomly selected for DNA sequencing. Of the 31 probe sets selected, 23 probe sets indicated detectable but consistently different expression levels for these genes in the two strains, and eight indicated similar expression levels in both strains. DNA sequencing was then performed on cDNA obtained from two different brain regions of both B6 and 129 mice. The number of true positives (TP), false positives (FP), true negatives (TN) and false negatives (FN) at various D-call thresholds was determined (see FIG. 7).

Using a D-call threshold of 1.8 increased the number of true positives while maintaining an acceptably low false positive rate: sixteen of twenty genes (80%) identified by the algorithm that were independently confirmed to have sequence differences. Of those sequenced, twelve contained sequence differences within their coding regions, leading to three changes in predicted amino acid sequence. In addition, all eleven of the genes flagged as identical between strains were confirmed to have no sequence differences.

The results indicate that an analysis of hybridization patterns obtained from gene expression measurements using oligonucleotide arrays can aid in the identification of genes that harbor genetic differences that may be responsible for interesting phenotypes. This generalized approach is directly applicable to the analysis of data obtained in expression studies in other organisms, with other array designs, and with different populations of mice. With current array designs, the sequence coverage for each gene may not be complete: 100 to 300 bases of sequence are interrogated for each gene because there are typically sixteen to twenty probe pairs per gene, the probes are 25 bases in length, some of the probes are overlapping, and sequence differences that result in mismatches near the probe ends (e.g., the five bases at either end) are not expected to lead to consistently measurable hybridization differences. Nonetheless, this approach allows an investigator to take advantage of previously existing data, obtained initially for other purposes, to search in a broad and unbiased way for genetic differences without the need for any additional experiments. While this analysis methodology has been described in terms of relatively small sequence differences (e.g., single-base substitutions), the approach can be used to identify larger differences (e.g., large deletions) and genes with different splice forms. Analyses of this type can be used to complement gene expression and other more traditional studies to accelerate the identification of genes that mediate important phenotypes.

Although the foregoing description of the invention has shown, described and pointed out novel features of the invention, it will be understood that various omissions, substitutions, and changes in the form of the detail of the apparatus as illustrated, as well as the uses thereof, may be made by those skilled in the art without departing from the spirit of the present invention. Consequently the scope of the invention should not be limited to the foregoing discussion but should be defined by the following claims.

What is claimed is:

1. A system for determining genetic differences between a first nucleotide population and a second nucleotide population, comprising:
a first detector module configured to read first gene expression data from a first expression array contacted by said first nucleotide population;
a second detector module configured to read second gene expression data from a second expression array contacted by said second nucleotide population, wherein the first expression array and the second expression array comprise a plurality of oligonucleotide fragments of an expressed gene sequence; and
a data processing module configured to compare the first gene expression data with the second gene expression data to calculate the genetic differences between the first nucleotide population and the second nucleotide population.

2. The system of claim 1, wherein the system is a personal computer system or workstation.

3. The system of claim 1, comprising a display module that graphically displays genetic differences between the first nucleotide population and the second nucleotide population.

4. The system of claim 1, wherein the first detector module and the second detector module are the same.

5. The system of claim 1, wherein the first expression array and the second expression array are the same.

6. The system of claim 5, wherein the first nucleotide population and the second nucleotide population are differentially labeled.

7. The system of claim 1, wherein the first nucleotide population and the second nucleotide population comprise DNA derived from expressed sequence templates.

8. The system of claim 1, wherein the first nucleotide population and the second nucleotide population comprise RNA derived from expressed sequence templates.

9. The system of claim 1, wherein the first nucleotide population is obtained from a first cell type and the second nucleotide population is obtained from a second cell type.

10. The system of claim 9, wherein the first cell type is derived from a first type of organism and the second cell type is derived from a second type of organism.

11. The system of claim 1, wherein the first nucleotide population and second nucleotide population are derived from different individuals.

12. The system of claim 1, wherein the first nucleotide population comprises genes related to disease conditions.

13. The system of claim 12, wherein the second nucleotide population comprises genes related to disease conditions.

14. A method for determining sequence variations using gene expression arrays wherein the gene expression arrays comprise a plurality of oligonucleotide fragments of one or more expressed genes, the method comprising:
analyzing a first nucleotide population using a first expression array to produce a first binding pattern;
analyzing a second nucleotide population using a second expression array to produce a second binding pattern; and
identifying differences between the first binding pattern and the second binding pattern to determine binding differences between the first nucleotide population and second nucleotide population.

15. The method of claim 14, further comprising normalizing the first and second binding patterns with respect to one another to account for expression level differences.

16. The method of claim 15, wherein normalizing the first and second binding patterns further comprises determining a scaling factor that is applied to the first and second binding patterns to generate first and second scaled binding patterns that are subsequently compared to distinguish sequence variations.

17. The method of claim 14, wherein the sequence variations of the first nucleotide population and the second nucleotide population result from analogous genes having different sequences between the nucleotide populations.

18. The method of claim 14, wherein the oligonucleotide fragments of the one or more expressed sequences comprise a plurality of probe pairs that contain a homologous probe having a known sequence and a partially homologous probe similar to the match probe but containing one or more sequence differences such that the nucleotide populations bind to the homologous match and partially homologous probes with differential affinity.

19. The method of claim 18, further comprising determining a differential affinity of binding between the homologous probe and the partially homologous probe that is used to identify sequence variations between the first nucleotide population and the second nucleotide population.

20. The method of claim 19, wherein the differential affinity of binding between the homologous probe and the partially homologous probe across the plurality of probe pairs is compared to identify binding pattern differences between the first and the second nucleotide populations.

21. The method of claim 14, wherein determining binding difference between oligonucleotide fragments and said first nucleotide population and oligonucleotide fragments and said second nucleotide population identifies polymorphisms in analogous genes of the first nucleotide population and the second nucleotide population.

22. The method of claim 14, wherein the first and second arrays are the same.

23. The method of claim 22, further comprising differentially labeling the first and second nucleotide populations to distinguish the first and second binding patterns from one another.

24. A method for identifying sequence variations between a first and second nucleotide population using gene expression arrays, the method comprising:
interacting the first nucleotide population with a first expression array to generate a first binding pattern;
interacting the second nucleotide population with a second expression array to generate a second binding pattern;
scaling the first and second binding patterns with respect to one another to create a first and second normalized binding pattern; and
identifying differences between the first and the second normalized binding patterns indicative of sequence variations between the first and the second nucleotide populations.

25. The method of claim 24, further comprising calculating a difference threshold which is applied to the first and second normalized binding patterns to identify sequence variations between the first and the second nucleotide populations.

26. The method of claim 25, wherein calculating the difference threshold further comprises determining an average binding pattern difference for the first and the second binding patterns.

27. The method of claim 26, wherein the difference threshold determines selectivity of the identification of sequence variations between the first and the second nucleotide populations.

28. A method for identifying sequence variations between a first and second nucleotide sequence using binding information obtained from oligonucleotide array analysis, the method comprising:
obtaining binding information from oligonucleotide array analysis using at least two nucleotides wherein a first nucleotide contains a first expressed nucleotide sequence having a first binding pattern and a second nucleotide contains a second expressed nucleotide sequence having a second binding pattern;
normalizing the binding patterns to produce a first scaled binding pattern and a second scaled binding pattern;
comparing the first scaled binding pattern and the second scaled binding pattern to identify differences between the scaled binding patterns; and
associating the differences with sequence variations between the first and the second expressed nucleotide sequence.

29. The method of claim 28, wherein scaled binding patterns account for differences in hybridization intensity such that the identified differences between the scaled binding patterns are representative of sequence variations.

30. The method of claim 28, further comprising identifying mutations in the first and the second expressed nucleotide sequence using the associated sequence variations.

31. The method of claim 28, further comprising identifying polymorphisms in the first and the second nucleotide sequence using the associated sequence variations.

32. The method of claim 28, further comprising associating the sequence variations with disease states.

33. The method of claim 28, further comprising identifying phenotypic differences between the organisms that provided the first and the second expressed nucleotide sequences that are associated with the sequence variations.

34. A method for identifying sequence variations using expression pattern differences, the method comprising:
identifying a first expression pattern for a first nucleotide strand and a second expression pattern for a second nucleotide strand wherein the first and second expression patterns are obtained by annealing the first and second nucleotide strands with a complimentary probe set;
comparing the first expression pattern and the second expression pattern and identifying differences between the binding of the first nucleotide strand with the complimentary probe set and the binding of the second nucleotide strand with the complimentary probe set; and
identifying sequence variations between the first nucleotide strand and the second nucleotide strand based upon the binding differences.

35. The method of claim 34, further comprising identifying mutations that are associated with the sequence variations between the first and the second nucleotide strands.

36. The method of claim 34, further comprising identifying polymorphisms that are associated with the sequence variations between the first and the second nucleotide strands.

37. The method of claim 34, further comprising identifying disease states that are associated with the sequence variations between the first and the second nucleotide strands.

38. The method of claim 34, further comprising identifying phenotypic differences arising from differences in sequence between the first and the second nucleotide strands.
